(19) United States
(12) Patent Application Publication
(10) Pub. No.: US 2024/0290463 A1
(43) Pub. Date: Aug. 29, 2024

(54) CLINICAL SUPPORT SYSTEM AND CLINICAL SUPPORT APPARATUS

(71) Applicant: CANON MEDICAL SYSTEMS CORPORATION, Otawara-shi (JP)

(72) Inventors: Atsuko SUGIYAMA, Nasushiobara (JP); Mariko SHIBATA, Nasushiobara (JP); Katsuhiko FUJIMOTO, Saitama (JP)

(73) Assignee: CANON MEDICAL SYSTEMS CORPORATION, Otawara-shi (JP)

(21) Appl. No.: 18/176,097

(22) Filed: Feb. 28, 2023

(51) Int. Cl. G16H 20/70; G16H 10/20; G16H 40/63

(52) U.S. Cl. CPC G16H 20/70; G16H 10/20; G16H 40/63

(57) ABSTRACT

A clinical support system according to an embodiment includes a clinical support apparatus, which is provided with processing circuitry, and a display device. The processing circuitry configured to acquire patient information related to a patient, interview information showing a response detail of the patient with respect to an interview, and conversation information recording a conversation of the patient; and the processing circuitry configured to generate patient characteristic information showing a mental aspect and a social aspect of the patient by an analyzing process using the patient information, the interview information, and the conversation information. The display device distinguishably displays the mental aspect and the social aspect of the patient based on the patient characteristic information.

### FIELD

Embodiments described herein relate generally to a clinical support system and a clinical support apparatus.

### BACKGROUND

One of the problems to be solved by embodiments disclosed in the present specification and drawings is to support medical practice appropriate for various aspects of a patient. However, the problems to be solved by the embodiments disclosed in the present specification and drawings are not limited to the above described problem. Problems corresponding to the effects of configurations shown in the later-described embodiments can be regarded as other problems.

### BRIEF DESCRIPTION OF THE DRAWINGS

FIG. 1 is a block diagram illustrating an example of a configuration of a clinical support system according to a present embodiment;

FIG. 2 is a block diagram illustrating an example of a configuration of a clinical support apparatus according to the present embodiment;

FIG. 3 is a diagram illustrating an example of a data configuration of analysis target information;

FIG. 4 is an explanatory diagram illustrating an example of a patient analyzing screen according to the present embodiment;

FIG. 5 is a flow chart illustrating an example of a display process executed by the clinical support apparatus according to the present embodiment;

FIG. 6 is an explanatory diagram illustrating an example of a patient analyzing screen according to Modification Example 1;

FIG. 7 is a diagram illustrating an example of a first category screen illustrating category items including a middle category according to Modification Example 8; and

FIG. 8 is a diagram illustrating an example of a second category screen according to Modification Example 8.

### DETAILED DESCRIPTION

Hereinafter, with reference to the drawings, a clinical support system and a clinical support apparatus related to the embodiment will be described. In the following embodiment, the parts denoted by the same reference signs are assumed to carry out similar operation, and redundant description thereof will be appropriately omitted.

### Present Embodiment

Medical workers such as doctors are required to carry out holistic medical care based on mental aspects and social aspects of patients in addition to biological aspects. In more detail, the medical workers are required to determine medical practice policies also in consideration of various aspects instead of determining medical practice policies only by biological aspects such as results of image diagnosis and results of clinical tests.

For example, patients with chronical conditions such as lifestyle-related diseases may not be able to heal only by the treatment of medication administration from biological aspects. For example, in a case of backache, the medical workers are preferred to give advice depending on the lifestyles and substances of work of patients in addition to prescription of a poultice.

When the medical workers carry out medical practices that encourage improvement in lifestyles, further improvement can be expected than the treatment that is only with medication administration. When the medical practice policies are determined also in consideration of whether the patient is with or without a child (children) and of the state of work, adherence (the extent that the patient is actively involved with treatment and medication and receives treatment in accordance with that determination) is improved during the treatment and after the treatment through the treatment appropriate for his/her lifestyle, and the patient can practice a better lifestyle and obtain a better outcome. Furthermore, since informing and treatment corresponding to the personality of the patient himself/herself is carried out, the patient can comfortably focus on the treatment.

In this manner, when the medical workers determine medical practice policies based on the biological aspects, the mental aspects, and the social aspects, the medical workers can provide better medical care. Therefore, the medical workers are required to provide so-called holistic medical care which also takes the mental aspects and the social aspects into consideration in addition to the biological aspects. Accordingly, the medical workers collect information related to the mental aspects and the social aspects by communicating with patients.

However, the medical workers communicate with the patients based on self-efforts and experiences. Also, it is sometimes difficult for the medical workers to collect the information related to the mental aspects and the social aspects. The medical workers also have to decide which information is to be recorded in electronic medical records or the like among the information related to the mental aspects and the social aspects. Furthermore, the medical workers are required to utilize the information from a higher perspective. Therefore, techniques that support medical practice appropriate for various aspects of the patient such as the mental aspect and the social aspects are desired.

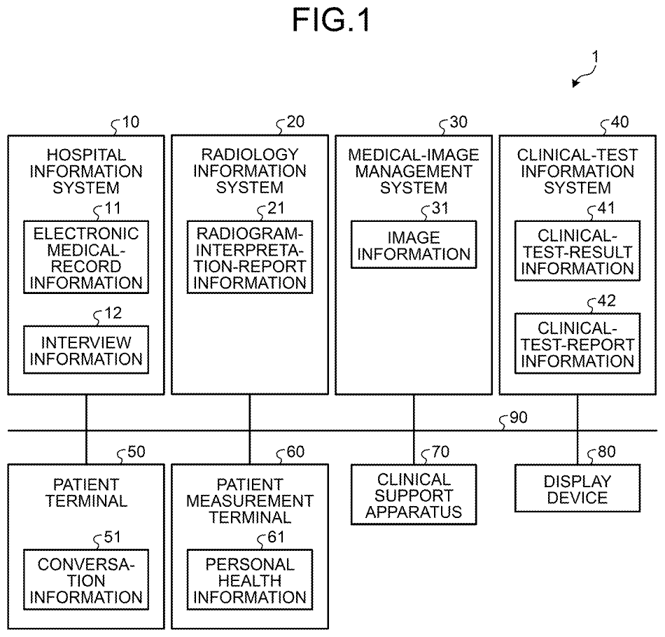

FIG. 1 is a block diagram illustrating an example of a configuration of a clinical support system 1 according to the present embodiment. The clinical support system 1 is provided with: a hospital information system (HIS: Hospital Information System) 10, a radiology information system (RIS: Radiology Information Systems) 20, a medical-image management system (PACS: Picture Archiving and Communication Systems) 30, a clinical-test information system (LIS: Laboratory Information System) 40, a patient terminal 50, a patient measurement terminal 60, a clinical support apparatus 70, and a display device 80. The systems and devices are communicably connected mutually via a network 90. Note that the configuration illustrated in FIG. 1 is an example, and the number of the systems and the devices may be arbitrary changed. Also, a device (s) not illustrated in FIG. 1 may be connected to the network 90.

The hospital information system 10, the radiology information system 20, the medical-image management system 30, and the clinical-test information system 40 are realized, for example, by computer equipment such as servers or workstations.

The hospital information system 10 stores electronic medical-record information 11 and interview information 12. The electronic medical-record information 11 is the information which records the courses of medical practice of patients. For example, the electronic medical-record information 11 has information such as the information for identifying the patients, personal information such as age, gender, and family members of the patients, the names of diseases of the patients, the names of prescribed medication, and periods of treatment. The interview information 12 is the information showing response details of the patients with respect to the interviews. For example, the interview information 12 has responses to the current symptoms of the patients, responses to the lifestyle environments of the patients, and responses to the personality diagnosis of the patients.

The radiology information system 20 stores radiogram-interpretation-report information 21. The radiogram-interpretation-report information 21 is the information which has opinions of doctors or the like who carried out the radiogram interpretation of image information 31 captured by medical image diagnosis apparatus.

The medical-image management system 30 stores the image information 31. The image information 31 has images captured by the medical image diagnosis apparatus. Examples of the medical image diagnosis apparatus include apparatus such as X-ray Computed Tomography (CT) apparatus, Magnetic Resonance Imaging (MRI) apparatus, X-ray diagnosis apparatus, and ultrasonic-wave diagnosis apparatus. The image information 31 follows the standard of Digital Imaging and Communications in Medicine (DICOM).

The clinical-test information system 40 stores clinical-test-result information 41 and clinical-test-report information 42. The clinical-test-result information 41 is the information showing the results of clinical tests. The clinical-test-report information 42 is the information which has opinions with respect to the clinical test results. The patient terminal 50 is a terminal used by the patient. The patient terminal 50 is realized, for example, by a smartphone, a tablet terminal, a wearable terminal, or a personal computer. The patient terminal 50 stores conversation information 51 in which conversations of the patient are recorded. For example, the conversation information 51 is the information of the conversations between medical workers such as doctors, nurses, and counselors and the patient. The conversation information 51 is not limited to the conversations related to medical practice details, but may include small talk. Specifically, the conversation information 51 has sound information which has recorded the conversations or the information which has transcribed the sound. The conversation information 51 may have video information such as moving images and still images including the conversations. If the video image is included, the conversation information 51 has a video which shows facial expressions of the patient.

The patient measurement terminal 60 is a terminal which measures vital signs of the patient. The patient measurement terminal 60 is realized, for example, by a wearable terminal. The patient measurement terminal 60 measures vital signs such as blood pressure, pulse, body temperature, and breathing. Also, the patient measurement terminal 60 may record conversations. The patient measurement terminal 60 stores personal health information 61 which has the measurement results of the vital signs.

Note that the patient measurement terminal 60 is not limited to a wearable terminal, but may be a body fat scale, an activity amount meter, or another device. Also, the patient measurement terminal 60 may measure body fat, may measure the amount of activity, or may measure another matter. Furthermore, the clinical support system 1 may be provided with plural patient measurement terminals 60. Also, in the clinical support system 1, the plural patient measurement terminals 60 may each have different measurement targets.

The clinical support apparatus 70 is a device which supports medical workers such as doctors in clinical practice. The clinical support apparatus 70 is realized, for example, by computer equipment such as a server or a workstation. The clinical support apparatus 70 analyzes the biological aspects, social aspects, and mental aspects of the patient based on the information acquired from the systems or devices of the clinical support system 1. Also, the clinical support apparatus 70 analyzes, as other aspects, the information not belonging to the biological aspects, the social aspects, or the mental aspects of the patient. Also, the clinical support apparatus 70 generates the information which shows the biological aspects, the social aspects, the mental aspects, and the other aspects of the patient. The clinical support apparatus 70 may be installed in a facility such as a hospital or may be a server on the Internet.

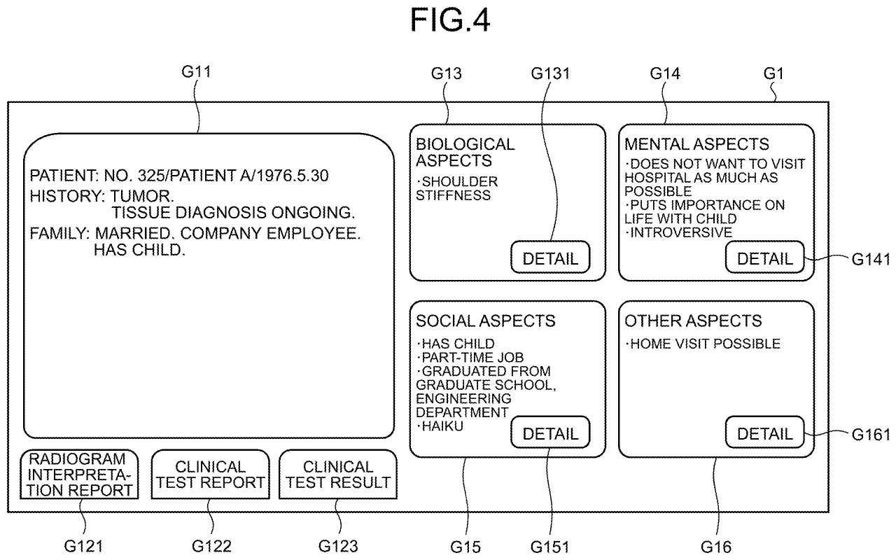

The display device 80 is a device which can display various information. The display device 80 is realized by computer equipment such as a personal computer or a tablet terminal. The display device 80 displays various information generated by the clinical support apparatus 70. For example, the display device 80 displays a patient analyzing screen G1 (see FIG. 4). The display device 80 is an example of a display control unit.

The medical worker such as a doctor can find out the social aspects and the mental aspects of the patient by seeing images displayed on the display device 80. Therefore, the medical worker can create treatment policies appropriate for the social aspects and the mental aspects of the patient.

Next, the clinical support apparatus 70 will be described.

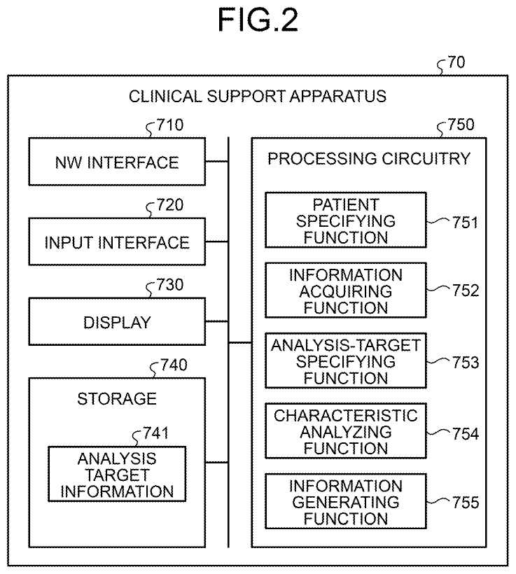

FIG. 2 is a block diagram illustrating an example of the configuration of the clinical support apparatus 70 according to the present embodiment. The clinical support apparatus 70 has a network (NW) interface 710, an input interface 720, a display 730, a storage 740, and a processing circuitry 750.

The NW interface 710 is connected to the processing circuitry 750 and controls transmission and communication of various data carried out with the devices connected via the network 90. For example, the NW interface 710 is realized by a network card, a network adapter, a Network Interface Controller (NIC), or the like.

The input interface 720 is connected to the processing circuitry 750, converts input operations which have been received from an operator (medical worker) to electric signals, and outputs the electric signals to the processing circuitry 750. Specifically, the input interface 720 converts the input operations, which have been received from the operator, to electric signals and outputs the electric signals to the processing circuitry 750. For example, the input interface 720 is realized by a trackball, switch buttons, a mouse, a keyboard, a touch pad which carries out input operations when an operation surface is touched, a touch screen integrating a display screen and a touch pad, a non-contact input circuit using optical sensors, a sound input circuit, and so on. Note that, in the present specification, the input interface 720 is not limited to that with physical operating parts such as a mouse and a keyboard. For example, examples of the input interface 720 also include a processing circuit of electric signals, which receives the electric signals corresponding to input operations from external input equipment provided separately from the device and outputs the electric signals to a control circuit.

The display 730 is connected to the processing circuitry 750 and displays various information and various image data output from the processing circuitry 750. For example, the display 730 is realized by a liquid-crystal display, a Cathode Ray Tube (CRT) display, an organic EL display, a plasma display, a touch panel, or the like.

The storage 740 is connected to the processing circuitry 750 and stores various data. Also, the storage 740 stores various programs for realizing various functions when the programs are read and executed by the processing circuitry 750. For example, the storage 740 is realized by a semiconductor memory element such as a Random Access Memory (RAM) and a flash memory, a hard disk, an optical disk, or the like.

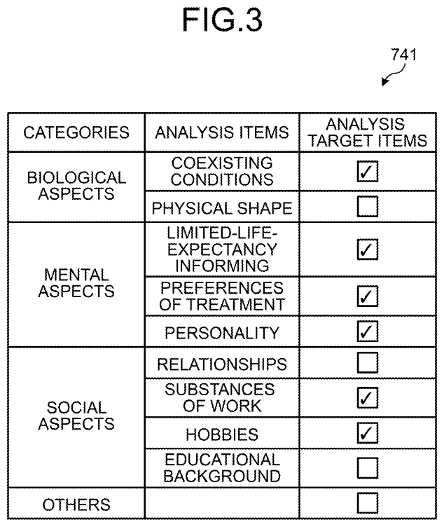

The storage 740 stores analysis target information 741. FIG. 3 is a diagram illustrating an example of a data configuration of the analysis target information 741. The analysis target information 741 is the information in which the targets to be analyzed as the biological aspects, the social aspects, the mental aspects, and the other aspects of the patient are specified among the information acquired by the systems and the devices of the clinical support system 1. In the analysis target information 741, categories, analysis items, and analysis target items are mutually associated. The categories are classifications of the biological aspects, the social aspects, the mental aspects, and so on. The biological aspects are the category which shows biological aspects of the patient. For example, the biological aspects are associated with biological analysis items such as various test results, conditions, and physical shape of the patient. Also, the biological aspects are not limited to the biological aspects, but may be associated with analysis items related to physical aspects, medical aspects, and pathological aspects. The mental aspects are the category which shows the mental aspects of the patient. For example, the mental aspects are associated with analysis items of the mental aspects such as the will and personality of the patient. Also, the mental aspects may include the emotional state of the patient. The emotional state represents emotions such as a depressive tendency or a tendency to be upset. The mental aspects may include limited-life-expectancy informing which shows whether the patient wants to know limited life expectancy or not, preferences of treatment which show how the patient make choices on treatment, thoughts and values of the patient, the personality of the patient, and so on. The social aspects are the category which shows the social aspects of the patient. For example, the social aspects are associated with analysis items of social aspects such as work, relationships, hobbies, educational background, and so on. The others are associated with the analysis items which do not belong to any of the biological aspects, the mental aspects, and the social aspects. The analysis items are the items for analyzing the patient. The analysis target items are the information in which whether the analysis items are to be analyzed or not is set. More specifically, the clinical support apparatus 70 analyzes the analysis items which are set to be analyzed among the analysis target items. The associating of the categories and the analysis items of the analysis target information 741 illustrated in FIG. 3 is an example and may be arbitrarily changed.

The processing circuitry 750 controls overall operation of the clinical support apparatus 70. The processing circuitry 750 has, for example, a patient specifying function 751, an information acquiring function 752, an analysis-target specifying function 753, a characteristic analyzing function 754, and an information generating function 755. In the embodiment, processing functions carried out by the patient specifying function 751, the information acquiring function 752, the analysis-target specifying function 753, the characteristic analyzing function 754, and the information generating function 755 which are constituent elements are stored in the storage 740 in the form of computer-executable programs. The processing circuitry 750 is a processor which realizes the functions corresponding to the programs by reading the programs from the storage 740 and executing the programs. In other words, the processing circuitry 750 in the state that the programs have been read have the functions illustrated in the processing circuitry 750 of FIG. 2.

Note that it has been described that, in FIG. 2, the single processor realizes the patient specifying function 751, the information acquiring function 752, the analysis-target specifying function 753, the characteristic analyzing function 754, and the information generating function 755. However, the functions may be realized by combining plural independent processors to build the processing circuitry 750 and executing the programs by the processors. Also, it has been described that, in FIG. 2, the single storage circuit such as the storage 740 stores the programs corresponding to the processing functions. However, plural storage circuits may be dispersedly disposed, and the processing circuitry 750 may be configured to read the programs from the individual corresponding storage circuits.

The word “processors” used in the above description means, for example, circuits such as Central Processing Units (CPUs), Graphical Processing Units (GPUs), Applications Specific Integrated Circuits (ASICs), programmable logic devices (for example, Simple Programmable Logic Devices: SPLDs) , Complex Programmable Logic Devices: CPLDs), Field Programmable Gate Arrays (FPGAs) , and so on. The processor (s) realizes the functions by reading and executing the programs saved in the storage 740. Note that, instead of saving the programs in the storage 740, the programs may be configured to be directly embedded in the circuit of the processor. In such a case, the processor realizes the functions by reading and executing the programs embedded in the circuit.

The patient specifying function 751 receives specifying of the patient who is the target for creating treatment policies. In other words, the patient specifying function 751 receives specifying of the patient who is the target for analyzing the biological aspects, the social aspects, and the mental aspects of the patient. The patient specifying function 751, for example, receives specifying of the patient from the display device 80. For example, the patient specifying function 751 receives the information which enables identifying of the patient such as the name of the patient and a patient code for distinguishing the patient. Also, when a choice is made from a list of patients, the patient specifying function 751 receives specifying of the patient.

The information acquiring function 752 acquires patient information related to the patient, the interview information 12 showing the response details of the patient with respect to interviews, and the conversation information 51 in which the conversations of the patient are recorded. The information acquiring function 752 is an example of an acquisition unit. Herein, the patient information is the information obtained by monitoring the patient such as the electronic medical-record information 11, the radiogram-interpretation-report information 21, the image information 31, the clinical-test-result information 41, the clinical-test-report information 42, and the personal health information 61. For example, when the patient is specified by the patient specifying function 751, the information acquiring function 752 acquires the patient information, the interview information 12, and the conversation information 51 of the specified patient.

The analysis-target specifying function 753 specifies the analysis target items which are the target analysis items to be analyzed by the characteristic analyzing function 754 among the analysis items of the biological aspects, the mental aspects, and the social aspects of the patient. The analysis-target specifying function 753 is an example of a specifying unit. In other words, the analysis-target specifying function 753 specifies the analysis target items of the analysis target information 741.

In more detail, the analysis-target specifying function 753 receives specifying of the analysis targets to be analyzed by using the patient information, the interview information 12, and the conversation information 51 acquired by the information acquiring function 752. For example, the analysis-target specifying function 753 receives specifying of the analysis targets by the information received by the NW interface 710 or by operation received by the input interface 720. For example, the analysis-target specifying function 753 receives specifying of the analysis targets from the display device 80 via the NW interface 710. Then, the analysis-target specifying function 753 specifies the analysis target items of the specified analysis target information 741 as analysis targets.

The characteristic analyzing function 754 generates patient characteristic information, which shows the biological aspects, the mental aspects, the social aspects, and the other aspects of the patient, by an analyzing process using the patient information, the interview information 12, and the conversation information 51. The characteristic analyzing function 754 is an example of an analysis unit. The analyzing process is a process of subjecting the patient information, the interview information 12, and the conversation information 51 to information extraction, determination, analysis, and generation of new information. The patient characteristic information is the information which shows characteristics of the patient in the biological aspects, the mental aspects, the social aspects, or the other aspects. Also, the patient characteristic information is the analysis results with respect to the analysis items of the biological aspects, the mental aspects, the social aspects, or the other aspects of the patient.

Herein, the characteristic analyzing function 754 generates the patient characteristic information which shows at least any one of the tendency for making decisions, preferences, and values of the patient as the mental aspects. In more detail, the characteristic analyzing function 754 generates the patient characteristic information which shows the tendency for making decisions in the cases where the patient decide things, the preferences showing what kind of options are preferred and selected by the patient, and the values of the patient as criterion to make decisions.

Also, the characteristic analyzing function 754 generates the patient characteristic information which shows at least any one of the social background and social environment of the patient as the social aspect. For example, factors of social determinants of health may serve as the social aspects. The social determinants of health are social factors which cause differences in the health state of individuals. The social determinants of health have a layered structure, and each layer has factors. For example, the social determinants of health include social background such as educational background, income, and occupation and social environments such as housing, public security, and traffic. Thus, the characteristic analyzing function 754 generates the patient characteristic information which shows the social background and/or social environment of the patient as the social determinants.

In more detail, the characteristic analyzing function 754 generates the patient characteristic information of the analysis target items of each of the biological aspects, the mental aspects, and the social aspects of the patient. The characteristic analyzing function 754 generates the patient characteristic information corresponding to the analysis target items of the biological aspect specified by the analysis target information 741 among the information included in the patient information. Specifically, the characteristic analyzing function 754 generates the patient characteristic information which shows coexisting conditions, physical shape, etc. included in the patient information.

Also, the characteristic analyzing function 754 generates the patient characteristic information of the analysis target items of the mental aspects and the social aspects specified by the analysis target information 741 among the information included in the patient information, the interview information 12, and the conversation information 51. For example, the characteristic analyzing function 754 generates the patient characteristic information corresponding to the analysis target items of the mental aspects and the social aspects specified by the analysis target information 741 among the interview information 12. Specifically, the characteristic analyzing function 754 extracts the responses to questions about personality determination, work, relationships, etc. included in the interview information 12.

Also, the characteristic analyzing function 754 generates the patient characteristic information including conversation detail information and emotion information of the conversations by analyzing the conversation information 51. The conversation detail information is the information which shows the details of the conversations. The emotion information is the information which shows the emotion of the patient when words in the conversations were spoken.

In more detail, the characteristic analyzing function 754 generates the patient characteristic information which includes the conversation detail information and the emotion information corresponding to the analysis target items of the mental aspects and the social aspects specified by the analysis target information 741. For example, the characteristic analyzing function 754 generates the conversation detail information which shows the details of the conversations by executing natural language processing with respect to the conversation information 51. For example, if the conversation information 51 includes a speech “I have a backache”, the characteristic analyzing function 754 generates a text “I have a backache”.

Also, the characteristic analyzing function 754 generates the emotion information which shows the emotions of the speaker patient. In this process, if the symptoms are getting worse, the patient often speak with negative emotions. On the other hand, if the symptoms are improving, the patient often speak with positive emotions. For example, the characteristic analyzing function 754 generates the emotion information which shows the emotions of the patient expressed by, for example, the tone of the voice, a falter, and nuance in the speech “I have a backache”. In this manner, the characteristic analyzing function 754 generates the patient characteristic information in which the conversation detail information is associated with the emotion information.

For example, the characteristic analyzing function 754 may utilize artificial intelligence such as AI (Artificial Intelligence). For example, the characteristic analyzing function 754 generates the emotion information, which shows the emotions of the speaker of the conversations included in the conversation information 51, by a learned model generated by machine learning. The learned model is generated by supervised learning in which the conversation information 51 is input as input-side training data, and the emotion information which shows the emotions of speakers is input as output-side training data. Note that the learned model is not limited to be generated by such machine learning, but may be generated by another method.

Also, the characteristic analyzing function 754 may generate the information which shows the emotion information and values of the patient, by comparing the conversation information 51 with other information such as the patient information or the interview information 12. For example, if there is a response in the interview information 12 that he/she has a non-sedentary job of middle intensity, and if there is a response in the conversation information 51 that he/she has a non-sedentary job of 6 hours, the characteristic analyzing function 754 can generate the information that shows the values of the patient that the patient recognizes 6 hours as middle intensity.

Also, as the time elapses, social circumstances and mental circumstances of the patient change. Therefore, the characteristic analyzing function 754 can repeatedly generate the patient characteristic information, and the characteristic analyzing function 754 may generate the patient characteristic information in every certain period of time or may generate the patient characteristic information for every medical-practice event such as testing or disclosure.

Note that the characteristic analyzing function 754 may generate the patient characteristic information, which shows the mental aspects and the social aspects, but is not limited to generate the information for all of the biological aspects, the mental aspects, the social aspects, and the other aspects of the patient.

The information generating function 755 generates the information which is to be displayed on the display device 80. Then, the display device 80 displays the various information generated by the information generating function 755. For example, the information generating function 755 generates the information, which distinguishably shows the mental aspects and the social aspects of the patient, based on the patient characteristic information. Then, the display device 80 distinguishably displays the mental aspects and the social aspects of the patient based on the patient characteristic information. The information generating function 755 is an example of an information generating unit. Furthermore, the information generating function 755 may generate the information which shows the biological aspects and other aspects in addition to the mental aspects and the social aspects. In other words, the information generating function 755 may generate the information which distinguishably shows the biological aspects, the mental aspects, and the social aspects of the patient, based on the patient characteristic information.

For example, the information generating function 755 generates the patient analyzing screen G1, which distinguishably shows the mental aspects and the social aspects of the patient, based on the patient characteristic information. FIG. 4 is an explanatory diagram illustrating an example of the patient analyzing screen G1 according to the present embodiment. The patient analyzing screen G1 has: patient profile information G11, a radiogram-interpretation-report button G121, a clinical-test-report button G122, a clinical-test-result button G123, biological-aspect information G13, mental-aspect information G14, social-aspect information G15, and other-aspect information G16.

The patient profile information G11 is the information which shows a profile of the patient. For example, the patient profile information G11 is the information which shows the number for identifying the patient, the name of the patient, the date of birth of the patient, the injury/disease history of the patient, the family members of the patient, etc. Also, the patient profile information G11 may include medical images of the patient which have been captured by a medical diagnostic device. Also, the patient profile information G11 is not required to display all the medical images of the patient, but may display, for example, typical medical images. Also, the medical images may be the medical images with lower resolution, may be downsized, or may be thumbnail images.

The radiogram-interpretation-report button G121 is a button which receives operation for displaying the radiogram-interpretation-report information 21. The clinical-test-report button G122 is a button which receives operation for displaying the clinical-test-report information 42. The clinical-test-result button G123 is a button which receives operation for displaying the clinical-test-result information 41.

The biological-aspect information G13 is the information which shows the patient characteristic information related to the biological aspects of the patient among the patient characteristic information generated by the characteristic analyzing function 754. For example, the biological-aspect information G13 is the information which shows characteristic keywords, etc. related to the biological aspects of the patient. Also, the biological-aspect information G13 has a detail button G131, which displays details of the biological aspects of the patient characteristic information.

The mental-aspect information G14 is the information which shows the patient characteristic information related to the mental aspects of the patient among the patient characteristic information generated by the characteristic analyzing function 754. For example, the mental-aspect information G14 is the information which shows characteristic keywords, etc. related to the mental aspects of the patient. Also, the mental-aspect information G14 has a detail button G141, which displays details of the mental aspects of the patient characteristic information.

The social-aspect information G15 is the information which shows the patient characteristic information related to the social aspects of the patient among the patient characteristic information generated by the characteristic analyzing function 754. For example, the social-aspect information G15 is the information which shows characteristic keywords, etc. related to the social aspects of the patient. Also, the social-aspect information G15 has a detail button G151, which displays details of the social aspects of the patient characteristic information.

The other-aspect information G16 is the information which shows the patient characteristic information related to the other aspects of the patient among the patient characteristic information generated by the characteristic analyzing function 754. For example, the other-aspect information G16 is the information which shows characteristic keywords, etc. related to the other aspects of the patient. Also, the other-aspect information G16 has a detail button G161, which displays details of the other aspects of the patient characteristic information.

Also, in the biological-aspect information G13, the mental-aspect information G14, the social-aspect information G15, and the other-aspect information G16, the information generating function 755 may generate the information which shows emotions in the speech of the analysis items of the biological aspects, the mental aspects, and the social aspects of the patient. For example, the information generating function 755 may generate the information which shows the emotions in the speech of the keywords of the biological-aspect information G13, the mental-aspect information G14, the social-aspect information G15, and the other-aspect information G16.

In more detail, the information generating function 755 may generate the information which shows the emotions in the speech, by expressing the conversation detail information included in the patient characteristic information based on emotion information. For example, when the emotions in the speech are to be expressed, the information generating function 755 presents the words which are indicated by the conversation detail information, by the colors identified by the emotion information, adds the numerical values which are identified by the emotion information, to the words indicated by the conversation detail information, adds the characters which are identified by the emotion information, to the words indicated by the conversation detail information, and/or adds images such as icons, which are identified by the emotion information, to the words indicated by the conversation detail information. By virtue of this, the medical workers can understand what kind of emotions have been involved in the conversations and in the speech of the corresponding words.

Also, the information generating function 755 generates the information which shows that there are time-course changes in the biological aspects, the mental aspects, the social aspects, and the other aspects of the patient, based on the patient characteristic information. Herein, the characteristic analyzing function 754 generates the patient characteristic information for each different timing by the analyzing process using the patient information, the interview information 12, and the conversation information 51 acquired at the different timing. In other words, the characteristic analyzing function 754 generates the patient characteristic information for every different timing, for example, for every certain period of time or for every medical-practice event. The information generating function 755 compares plural pieces of the patient characteristic information of different timings. Then, if there is a change(s) between the plural pieces of patient characteristic information, the information generating function 755 generates the information which shows that there are time-course changes regarding the corresponding analysis items of the biological aspects, the mental aspects, the social aspects, and the other aspects.

The information generating function 755 generates the biological-aspect information G13, the mental-aspect information G14, the social-aspect information G15, and the other-aspect information G16 which show the presence of the time-course changes. For example, the information generating function 755 subjects the analysis items with the time-course changes to color changes, addition of the characters which indicate the presence of the time-course changes, or addition of images such as icons which indicate presence of the time-course changes.

Also, the information generating function 755 may generate the biological-aspect information G13, the mental-aspect information G14, the social-aspect information G15, and the other-aspect information G16 with which profiles of the time-course changes can be distinguished. For example, the information generating function 755 may generate the biological-aspect information G13, the mental-aspect information G14, the social-aspect information G15, and the other-aspect information G16 which expresses positive or negative emotional changes by colors, numerical values, characters, or images such as icons.

Also, the information generating function 755 may generate the information which comparably shows the analysis items which have undergone time-course changes. For example, when the detail button G131, G141, G151, or G161 is pressed, the information generating function 755 generates the information in which the analysis items of the patient characteristic information before the time-course change and the analysis items of the patient characteristic information after the time-course change are juxtaposed. By virtue of this, the medical worker can find out how the circumstances of the patient have changed.

Next, various processing executed by the clinical support system 1 will be described.

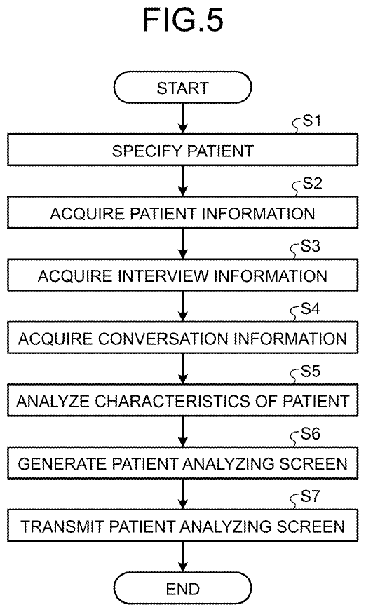

FIG. 5 is a flow chart illustrating an example of a display process executed by the clinical support apparatus 70 according to the present embodiment. The display process is a process in which the characteristic matters of the patient in the aspects are displayed.

The patient specifying function 751 receives specifying of a patient to be a target of the patient analyzing screen G1 (step S1).

The information acquiring function 752 acquires the patient information such as the electronic medical-record information 11, the radiogram-interpretation-report information 21, the image information 31, the clinical-test-result information 41, the clinical-test-report information 42, the personal health information 61, and the like (step S2).

The information acquiring function 752 acquires the interview information 12 (step S3).

The information acquiring function 752 acquires the conversation information 51 (step S4).

The characteristic analyzing function 754 generates the patient characteristic information which shows the biological aspects, the mental aspects, the social aspects, and other aspects of the patient by an analyzing process using the patient information, the interview information 12, and the conversation information 51 (step S5).

The information generating function 755 generates the patient analyzing screen G1 based on the patient characteristic information (step S6).

The information generating function 755 transmits the generated patient analyzing screen G1 to the display device 80 (step S7). As a result, the display device 80 displays the patient analyzing screen G1.

Thus, the clinical support apparatus 70 ends the display process.

As described above, the clinical support system 1 according to the embodiment acquires the patient information which shows monitoring results of the patient, the interview information 12, and the conversation information 51. Also, the clinical support system 1 acquires the patient characteristic information which shows the mental aspects and the social aspects of the patient, by the analyzing process using the patient information, the interview information 12, and the conversation information 51. Then, the clinical support system 1 generates the patient analyzing screen G1, which shows the mental aspects and the social aspects of the patient, based on the patient characteristic information. Then, the display device 80 displays the patient analyzing screen G1. By virtue of this, the medical worker can find out various aspects of the patient by the patient analyzing screen G1. Therefore, the clinical support system 1 can support the medical practice appropriate for the various aspects of the patient.

Furthermore, the medical worker can understand the patient from the biological aspects, the mental aspects, and the social aspects and examine the treatment policies in consideration of them. Therefore, the clinical support system 1 can promote holistic patient-centered medical care.

### Modification Example 1

In the present embodiment, it has been described that the information generating function 755 generates the patient analyzing screen G1 illustrated in FIG. 4. The information generating function 755 may generate a patient analyzing screen G2, which is in a form different from the patient analyzing screen G1 illustrated in FIG. 4. Then, the display device 80 may display the patient analyzing screen G2.

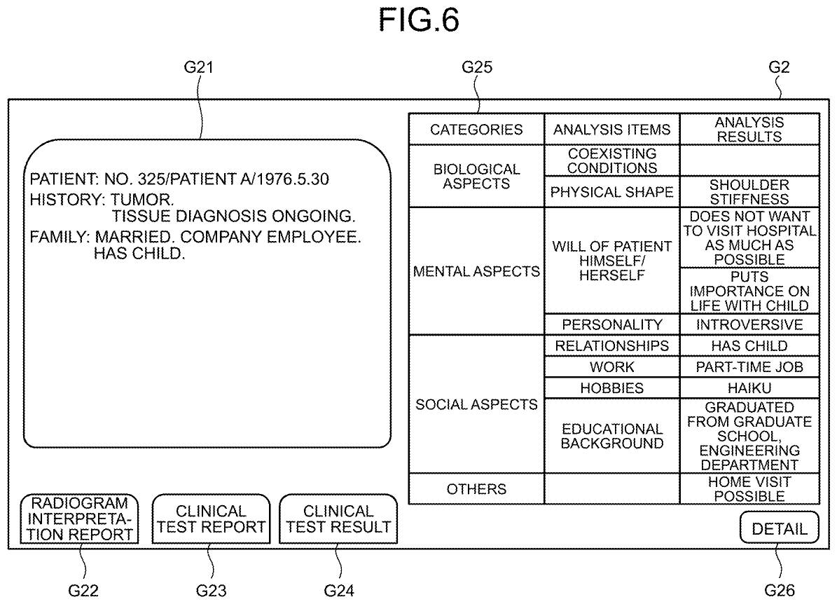

FIG. 6 is an explanatory diagram illustrating an example of the patient analyzing screen G2 according to Modification Example 1. Patient profile information G21, a radiogram-interpretation-report button G22, a clinical-test-report button G23, and a clinical-test-result button G24 are the information similar to the patient profile information G11, the radiogram-interpretation-report button G121, the clinical-test-report button G122, and the clinical-test-result button G123 of the patient analyzing screen G1 illustrated in FIG. 4. Also, the patient profile information G21 may include medical images of the patient which have been captured by a medical diagnostic imaging device. Also, the patient profile information G21 is not required to display all the medical images of the patient, but, for example, can display typical medical images. Also, the medical images may be medical images with lowered resolution, may be downsized, or may be thumbnail images.

Also, the information generating function 755 generates the patient analyzing screen G2 which has list information G25 instead of the biological-aspect information G13, the mental-aspect information G14, the social-aspect information G15, and the other-aspect information G16 of the patient analyzing screen G1 illustrated in FIG. 4.

The information generating function 755 generates the list information G25, which shows the patient characteristic information for every category and analysis item of the analysis target information 741. More specifically, the information generating function 755 generates the list information G25 which combines, into one group, the biological-aspect information G13, the mental-aspect information G14, the social-aspect information G15, and the other-aspect information G16. Also, the information generating function 755 has a detail button G26, which displays details of the biological aspects, the mental aspects, the social aspects, and the other aspects of the patient characteristic information. Moreover, as well as the above described embodiment, in the list information G25, the information generating function 755 may generate the information which shows the mental conditions in the corresponding speech and/or may generate the information which shows time-course changes. Also, when the detail button G26 is pressed, the information generating function 755 may generate the information which comparably shows the analysis items, which have undergone time-course changes.

### Modification Example 2

In the present embodiment, the information generating function 755 generates the patient analyzing screens G1 and G2. It has been described that the display device 80 displays the patient analyzing screens G1 and G2. The information generating function 755 may cause the patient analyzing screens G1 and G2 to be displayed on the display 730 of the clinical support apparatus 70 or on another device (s).

### Modification Example 3

In the present embodiment, it has been described that the information acquiring function 752 acquires the patient information, the interview information 12, and the conversation information 51. Furthermore, the information acquiring function 752 may acquire location information from the patient terminal 50, may acquire brain-wave information from the patient measurement terminal 60, or may acquire both of the information.

The location information is the information which shows the location of the patient terminal 50. In other words, the location information is the information which shows the location of the patient. The characteristic analyzing function 754 can extract commuting time, for example, by measuring the travel time from the home of the patient to his/her workplace based on the location information and map information at corresponding time. By a similar method, the characteristic analyzing function 754 can extract the time taken to visit the hospital. Furthermore, the characteristic analyzing function 754 can extract, for example, if the patient is active or if the patient tends to retreat at home based on the location information.

The brain-wave information is the information which shows the brain waves of the patient. For example, the information acquiring function 752 acquires the brain-wave information which shows the brain waves of the patient during conversations with the medical worker. By virtue of this, the characteristic analyzing function 754 can acquire the mental conditions of the patient during the conversations with the medical worker. For example, the characteristic analyzing function 754 can extract, for example, a fact about whether the patient is relaxed or is feeling stressed.

### Modification Example 4

The information generating function 755 may generate the information which shows standard treatment in the patient analyzing screen G1, G2, and/or the like. The standard treatment is the standard treatment which is carried out appropriately for injuries/diseases and the progress of the injuries/diseases.

In more detail, the information acquiring function 752 acquires standard treatment information, which shows the standard treatment appropriate for the injury/disease of the patient specified by the patient specifying function 751 and the progress of the injury/disease, from standard-treatment list information, which is a list of the standard treatment registered for each of injuries/diseases and the progress of the injuries/diseases. Then, the information generating function 755 generates the patient analyzing screen G1 or G2, which has the standard treatment shown by the standard treatment information, based on the standard treatment information. By virtue of this, the medical worker can create treatment policies appropriate for the mental aspects and the social aspects of the patient after checking the standard treatment, which is appropriate for the injury/disease of the patient and the progress of the injury/disease.

### Modification Example 5

The information generating function 755 may generate the information which shows the information of a similar patient (s) who is a patient (s) similar to the patient specified by the patient specifying function 751. For example, the information generating function 755 may generate the information which shows the electronic medical-record information 11 of the similar patient. For example, the information generating function 755 may generate the information which shows the electronic medical-record information 11 of a similar patient who is similar to the patient who has introversive personality and has a child. By virtue of this, the medical worker can take the treatment of the similar patient into consideration.

For example, the storage 740 stores the patient characteristic information of plural patients. The information generating function 755 calculates the degrees of similarity between the patient characteristic information generated by the characteristic analyzing function 754 and the patient characteristic information of the plural patients stored in the storage 740. Also, the information generating function 755 generates the patient characteristic information of the similarity degrees which are equal to or higher than a threshold value. The information generating function 755 determines the patient (s) of the generated patient characteristic information as a similar patient (s). Then, when the operation to display the information about the similar patient selected from the single or plural similar patients is received, the information generating function 755 generates the information which shows the specified information.

Note that the information generating function 755 may set importance degrees with respect to the categories and analysis items of the patient characteristic information and calculate similarity degrees. For example, if the importance degree of the social aspects is set to be high, the information generating function 755 selects the patient who has similar social aspects, as a similar patient. By virtue of this, the information generating function 755 can generate the information which shows the information of the patient who has similar categories or analysis items determined by the medical worker to be important.

### Modification Example 6

It has been described that the characteristic analyzing function 754 analyzes the characteristics of the biological aspects, the mental aspects, the social aspects, and the other aspects of the patient. More specifically, it has been described that the characteristic analyzing function 754 analyzes the speech of the patient in the conversation information 51. However, if the speech of the patient alone is displayed, the medical worker sometimes cannot find out the reason why the patient spoke like that. Therefore, the characteristic analyzing function 754 extracts the speech of the person other than the patient in the conversation information 51. In such a case, if the speaker is not clearly shown, the medical worker cannot find out who spoke the words.

If the speaker can be identified by subjecting the extracted voice to voiceprint collation, the characteristic analyzing function 754 may extract the speech details and the speaker. Also, if the speaker cannot be identified, the characteristic analyzing function 754 may extract information such as “SPEAKER A”, which shows that a person different from the patient has spoken, and speech details. Then, the information generating function 755 may generate the keywords indicated by the speech details and generate the information which shows the speaker who spoke the speech details in the patient analyzing screen G1 or G2.

By virtue of this, the medical worker who has seen the patient analyzing screen G1 or G2 can find out how the speech has been made. Furthermore, the medical worker can find out who spoke the words.

### Modification Example 7

The information generating function 755 generates the information which shows the analysis items for which the patient characteristic information has not been generated among the analysis target items specified by the analysis-target specifying function 753. In other words, the information generating function 755 generates the information which shows the analysis items for which the patient characteristic information has not been generated among the analysis target items of the analysis target information 741. Thus, the display device 80 displays the information which shows the analysis items for which the patient characteristic information has not been generated.

For example, the information generating function 755 may generate the information which shows the analysis items for which information has not been generated among the analysis items selected as analysis targets in the patient analyzing screen G1 or G2. By virtue of this, in medical attention or the like, the medical worker can ask the patient about the analysis items for which information has not been extracted. Therefore, the information generating function 755 can urge collection of the information about the analysis items for which information has not been generated.

### Modification Example 8

It has been described that the information generating function 755 generates the patient analyzing screen G1, which distinguishably shows the mental aspects and the social aspects of the patient as illustrated in FIG. 4. The information generating function 755 is not limited to generate the patient analyzing screen G1, but may generate a screen in which the mental aspects and the social aspects of the patient are categorized into the will of the patient, peripheral matters, Quality of Life (QOL), etc. The display device 80 may display the screen which has been generated by the information generating function 755.

Herein, the characteristic analyzing function 754 generates the patient characteristic information, which shows the biological aspects, the mental aspects, the social aspects, and other aspects of the patient, by an analyzing process using the patient information, the interview information 12, and the conversation information 51. Furthermore, the characteristic analyzing function 754 may categorize the information of the biological aspects, the mental aspects, the social aspects, and the other aspects included in the patient characteristic information into the items determined in advance. Also, the characteristic analyzing function 754 may categorize the information of the biological aspects, the mental aspects, and the social aspects included in the patient characteristic information, for example, into the will of the patient, peripheral matters, QOL, etc. In other words, the characteristic analyzing function 754 may generate the patient characteristic information in which the mental aspects and the social aspects of the patient are categorized by category items determined in advance. Furthermore, the characteristic analyzing function 754 may categorize the category items such as the will of the patient, peripheral matters, QOL, etc. gradually, for example, into a large category, a middle category, and a small category.

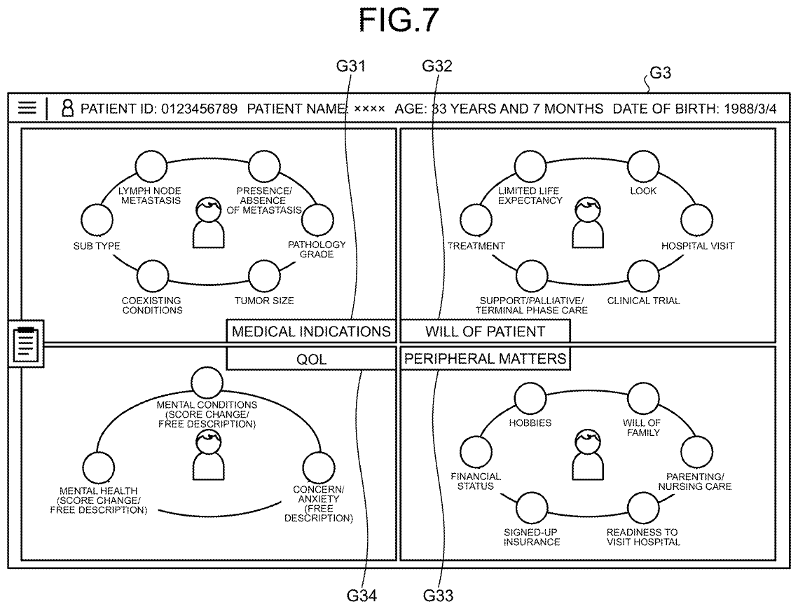

The information generating function 755 generates a screen which displays the patient characteristic information gradually by the large category, middle category, and small category regarding the will of the patient, peripheral matters, and QOL. FIG. 7 is a diagram illustrating an example of a first category screen G3 showing category items including the middle category according to Modification Example 8. The first category screen G3 has display areas which display category items of the middle category of medical indications, the will of the patient, peripheral matters, and QOL. The display areas may display all of the category items of the middle category or may display, among the category items of the middle category, the items which are associated with the information included in the patient characteristic information.

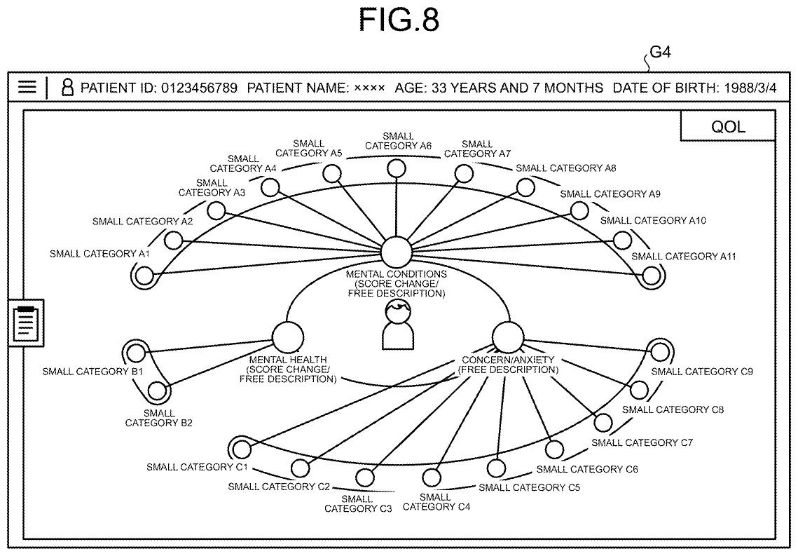

Also, the first category screen G3 has a medical-indication button G31, a patient-will button G32, a peripheral-matter button G33, and a QOL button G34. The medical-indication button G31, the patient-will button G32, the peripheral-matter button G33, and the QOL button G34 are buttons which receive operation for displaying a second category screen G4 (see FIG. 8), which shows category items including the small category included in the corresponding large category.

When any of the medical-indication button G31, the patient-will button G32, the peripheral-matter button G33, and the QOL button G34 is selected, the information generating function 755 generates the second category screen G4 of the corresponding large category. FIG. 8 is a diagram illustrating an example of the second category screen G4 according to Modification Example 8. The second category screen G4 has category items of the small category outside the category items of the middle category. The category items of the middle category and the category items of the small category are coupled by outgoing lines. By virtue of this, the second category screen G4 clearly shows the relations between the middle category and the small category and clearly shows to which category items of the middle category the category items of the small category belong. Then, the display device 80 generates screens such as the first category screen G2, the second category screen G4, etc. showing the mental aspects and the social aspects of the patient by the category items based on the patient characteristic information.

In this manner, the information generating function 755 generates the first category screen G3 and the second category screen G4 as the screens in which the patient characteristic information including the mental aspects and the social aspects of the patient has been categorized into medical indications, the will of the patient, the peripheral matters, QOL, etc. Then, the display device 80 displays the first category screen G3 and the second category screen G4. Therefore, the medical worker is facilitated to find out the mental aspects and the social aspects of the patient in a meeting for determining treatment policies, a meeting with medical workers of plural professions, or in medical attention. Therefore, the information generating function 755 can carry out support to advance medical care in consideration of the mental aspects and the mental aspects in addition to the biological aspects.

According to the at least one embodiment, etc. described above, the medical care appropriate for various aspects of the patient can be supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A clinical support system comprising: a clinical support apparatus; and a display device, the clinical support apparatus having processing circuitry, wherein
the processing circuitry configured to:
acquire patient information related to a patient, interview information showing a response detail of the patient with respect to an interview, and conversation information recording a conversation of the patient, and
generate patient characteristic information showing a mental aspect and a social aspect of the patient by an analyzing process using the patient information, the interview information, and the conversation information, and
the display device
distinguishably displays the mental aspect and the social aspect of the patient based on the patient characteristic information.

2. The clinical support system according to claim 1, wherein
the processing circuitry configured to generate the patient characteristic information showing at least any one of a tendency for making a decision, a preference, and a value of the patient as the mental aspect.

3. The clinical support system according to claim 1, wherein
the processing circuitry configured to generate the patient characteristic information showing at least any one of social background or a social environment of the patient as the social aspect.

4. The clinical support system according to claim 1, wherein
the processing circuitry configured to generate the patient characteristic information showing a biological aspect, the mental aspect, and the social aspect of the patient, and
the display device distinguishably displays the biological aspect, the mental aspect, and the social aspect of the patient based on the patient characteristic information.

5. The clinical support system according to claim 1, wherein
the processing circuitry configured to generate the patient characteristic information categorized by a category item determined in advance for the mental aspect and the social aspect of the patient, and
the display device displays the mental aspect and the social aspect of the patient by the category item based on the patient characteristic information.

6. The clinical support system according to claim 1, wherein
the display device displays that the biological aspect, the mental aspect, and the social aspect of the patient has a time-course change based on the patient characteristic information.

7. The clinical support system according to claim 6, wherein
the display device comparably displays an analysis item that has undergone the time-course change.

8. The clinical support system according to claim 1, wherein
the display device displays an emotion in speech of an analysis item of each of the biological aspect, the mental aspect, and the social aspect of the patient.

9. The clinical support system according to claim 1, wherein
the processing circuitry configured to:
specify, among the analysis item of each of the biological aspect, the mental aspect, and the social aspect of the patient, an analysis target item serving as the analysis item of an analysis target, and
generate the patient characteristic information of the analysis target item of each of the biological aspect, the mental aspect, and the social aspect of the patient.

10. The clinical support system according to claim 9, wherein
the display device displays the analysis item, among the analysis target item, the patient characteristic information having not been generated for the analysis item.

11. A clinical support apparatus comprising following processing circuitry, wherein
the processing circuitry configured to:
acquire patient information related to a patient, interview information showing a response detail of the patient with respect to an interview, and conversation information recording a conversation of the patient,
generate patient characteristic information showing a mental aspect and a social aspect of the patient by an analyzing process using the patient information, the interview information, and the conversation information, and
generate information distinguishably showing the mental aspect and the social aspect of the patient based on the patient characteristic information.
